(19) United States
(12) Patent Application Publication
(10) Pub. No.: US 2014/0148734 A1
(43) Pub. Date: May 29, 2014

(54) COLONOSCOPY SYSTEMS AND METHODS

(71) Applicants: Lyndon V. Hernandez, Franklin, WI (US); George K. Lewis, Ithaca, NY (US); Joseph Kost, Omer (IL)

(72) Inventors: Lyndon V. Hernandez, Franklin, WI (US); George K. Lewis, Ithaca, NY (US); Joseph Kost, Omer (IL)

(21) Appl. No.: 13/820,368

(22) PCT Filed: Sep. 1, 2011
(86) PCT No.: PCT/US11/50219

§ 371 Date: May 22, 2013

Related U.S. Application Data
(60) Provisional application No. 61/380,065, filed on Sep. 3, 2010.

(51) Int. Cl. A61N 7/02

(52) U.S. Cl. CPC A61N 7/022
USPC 601/2

(57) ABSTRACT

Systems and methods for laxative-free colonoscopy include an ultrasound transducer housing positioned at or near the operable end of the flexible tube, the housing including an ultrasound transducer to generate ultrasound energy. The flexible tube with ultrasound transducer is inserted into a gastrointestinal tract. A water flow channel delivers water to the gastrointestinal tract. The ultrasound energy and water liquefy stool in the gastrointestinal tract, and the liquefied stool is removed from the gastrointestinal tract.

### CROSS-REFERENCE TO RELATED APPLICATIONS

The present application claims priority to U.S. Provisional Patent Application No. 61/380,065, filed on Sep. 3, 2010 and entitled “A Method of Laxative-Free Colonoscopy,” which is incorporated herein by reference in its entirety.

### STATEMENT REGARDING FEDERALLY FUNDED RESEARCH OR DEVELOPMENT

Not Applicable

### FIELD OF THE INVENTION

The present invention is directed to ultrasound-based, laxative-free or laxative-failed systems and methods for performing colonoscopies.

### BACKGROUND OF THE INVENTION

Colon cancer is the second leading cause of cancer-related deaths in Western nations and can be prevented with a screening colonoscopy. Although there are emerging technologies such as computed-tomographic colonography and video capsule technology, colonoscopy combines diagnosis and treatment in one session by its ability to remove precancerous polyps, and is expected to remain the most dominant form of screening for several years to come.

Of the 70 million Americans over 50 years of age eligible for screening, 60% have never undergone any type of screening for colon cancer. One of the most important reasons why patients avoid this test is because of the required bowel cleansing (or prep) prior to colonoscopy, which is inconvenient, time-consuming, and generally unpleasant. In addition, the current methods of cleansing can cause abdominal cramping, nausea, vomiting, electrolyte imbalance, and renal failure. To the best of our knowledge, there is no bowel preparation solution on the market or in the pipeline that addresses these problems. Removing the laxative prep offers compelling opportunity to improve patient compliance. This point was driven home in a recent survey of Minnesota residents, in which 86% of the respondents would more likely undergo testing if the laxative prep were removed from the procedure altogether.

In addition to the above mentioned weaknesses of current colon preparation methods, poor colon cleansing also increases the duration of the colonoscopy by about 10% and the cost of the procedure by up to 22%. Poor colon cleansing occurs in about a quarter of colonsocopies performed each year in the United States. The increased cost is due to aborted and inadequate examinations that results from inadequate bowel cleansing. Inadequate bowel cleansing can occur when the patient cannot tolerate the laxative and was not able to finish the prep, or the prep was consumed but is not totally effective (i.e., the laxative-failed). Such situations require that patients return at an earlier interval for a repeat colonoscopy. In addition, poor colon cleansing before or during a colonoscopic examination leads to higher rates of missed precancerous polyps.

Current colonoscopies can irrigate the colonic lumen with water during a procedure and evacuate the fluids and unwanted debris using suction applied though the instrument working channel. However, these suction ports are inadequate when the physician is facing a poorly prepped patient with solid stools that cannot be aspirated.

It would, therefore, be desirable to provide a colonoscopy that avoids the undesirable effects of bowel cleansing prior to a traditional colonoscopy, or can salvage poorly cleansed colons from being aborted.

### SUMMARY OF THE INVENTION

The subject matter disclosed herein relates generally to systems and methods for performing a colonoscopy, and, more particularly, for performing a colonoscopy using ultrasound to cleanse the colon.

Systems and methods for laxative-free or laxative-failed colonoscopy include an ultrasound transducer housing positioned at or near the operable end of the flexible tube, the housing including an ultrasound transducer to generate ultrasound energy. The flexible tube with ultrasound transducer is inserted into a gastrointestinal tract. A water flow channel delivers water to the gastrointestinal tract. The ultrasound energy and water liquefy stool in the gastrointestinal tract, and the liquefied stool is removed from the gastrointestinal tract.

According to some embodiments, a medical device is provided. The device includes a flexible tube having an operable end insertable into a body cavity and a control end. An ultrasound transducer housing is positioned at or near the operable end of the flexible tube, the housing including an ultrasound transducer to generate ultrasound energy and at least one water flow channel to deliver water to the body cavity. An ultrasound generator circuit is coupled to the ultrasound transducer to control the ultrasound transducer.

According to other embodiments, a method is provided. The method includes the steps of introducing a flexible tube having an operable end insertable into a gastrointestinal tract, the flexible tubing including an ultrasound transducer housing positioned at or near the operable end of the flexible tube, the housing including an ultrasound transducer to generate ultrasound energy and at least one water flow channel to deliver water to the gastrointestinal tract; applying ultrasound energy to stool located in the gastrointestinal tract; delivering water through the at least one water flow channel to the gastrointestinal tract; liquefying the stool via the ultrasound energy and water; and removing the liquefied stool from the gastrointestinal tract.

The foregoing features and advantages of the invention will appear in the detailed description which follows. In the description, reference is made to the accompanying drawings that illustrate preferred embodiments and wherein like reference numerals denote like elements.

### BRIEF DESCRIPTION OF THE DRAWINGS

FIG. 1 shows a front perspective view of a colonoscopy device including an ultrasound transducer, in accordance with embodiments of the invention;

FIG. 2 shows a side perspective view of a portion of the colonoscopy device shown in FIG. 1, in accordance with embodiments of the invention;

FIG. 3 is an anatomical view showing an embodiment of the colonoscopy device of FIG. 1 positioned in a gastrointestinal tract for liquefaction of stool;

FIGS. 4A-C show a demonstration of stool liquefaction, in accordance with embodiments of the invention;

FIG. 5 shows an alternative embodiment of a colonoscopy device that may be swallowed by the subject; and

FIG. 6 is a flow chart of a method of use of an embodiment of the colonoscopy device of FIG. 1.

The invention may be embodied in several forms without departing from its spirit or essential characteristics. The scope of the invention is defined in the appended claims, rather than in the specific description preceding them. All embodiments that fall within the meaning and range of equivalency of the claims are therefore intended to be embraced by the claims.

### DETAILED DESCRIPTION

The following discussion is presented to enable a person skilled in the art to make and use embodiments of the invention. Various modifications to the illustrated embodiments will be readily apparent to those skilled in the art, and the generic principles herein can be applied to other embodiments and applications without departing from embodiments of the invention. Thus, embodiments of the invention are not intended to be limited to embodiments shown, but are to be accorded the widest scope consistent with the principles and features disclosed herein. The following detailed description is to be read with reference to the figures. The figures depict selected embodiments and are not intended to limit the scope of embodiments of the invention. Skilled artisans will recognize the examples provided herein have many useful alternatives and fall within the scope of embodiments of the invention.

The following description refers to elements or features being “connected” or “coupled” together. As used herein, unless expressly stated otherwise, “connected” means that one element/feature is directly or indirectly connected to another element/feature, and not necessarily mechanically. Likewise, unless expressly stated otherwise, “coupled” means that one element/feature is directly or indirectly coupled to another element/feature, and not necessarily mechanically. Thus, although embodiments shown in the figures depict example arrangements of colonoscopy devices, additional intervening elements, devices, features or components may be present in an actual embodiment.

In accordance with the practices of persons skilled in the art of computer programming, the present disclosure may be described herein with reference to operations that may be performed by various computing components, modules, or devices. Such operations may be referred to as being computer-executed, computerized, software-implemented, or computer-implemented. It will be appreciated that operations that can be symbolically represented include the manipulation by the various microprocessor devices of electrical signals representing data bits at memory locations in the system memory, as well as other processing of signals. The memory locations where data bits are maintained are physical locations that have particular electrical, magnetic, optical, or organic properties corresponding to the data bits.

The various aspects of the invention will be described in connection with laxative-free colonoscopy devices. That is because the features and advantages that arise due to embodiments of the invention are well suited to this purpose. Still, it should be appreciated that the various aspects of the invention can be applied to achieve other objectives as well.

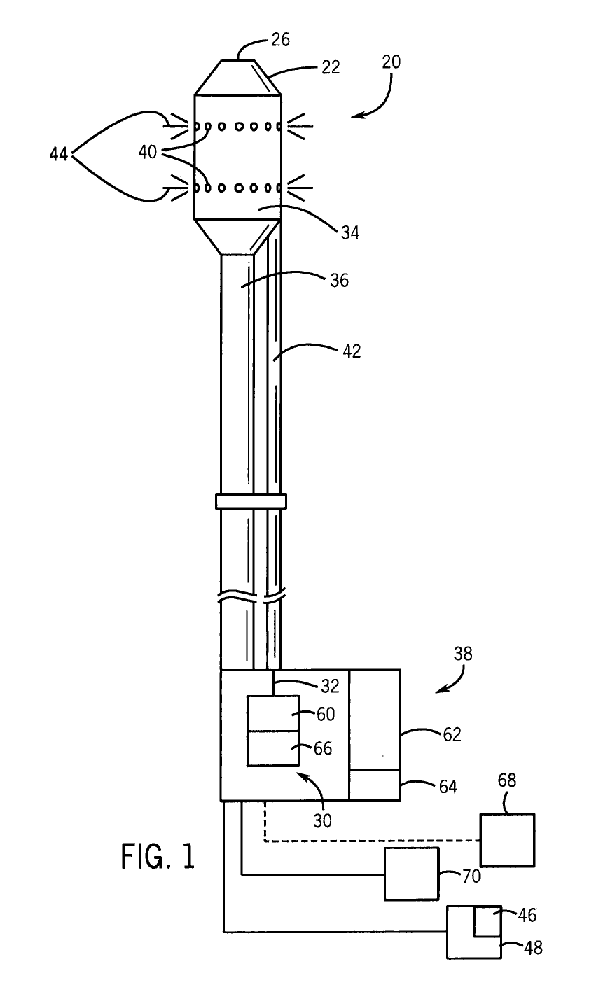

To address the aforementioned weaknesses and encourage increased compliance for undergoing colonoscopies, the present invention provides a colonoscope 20 with an ultrasound transducer 22 (see FIGS. 1, 2 and 3). The physician may use the ultrasound transducer 22 to liquefy stool(s) during the examination. The liquid stools can then be suctioned out and the examination continues. Standard colonoscopies usually deliver water in sufficient amounts and have a suction capability. Therefore, embodiments of the present invention provide a transducer designed to work with these systems. Our study shows that it is reasonable to expect to use a device to safely liquefy and suction stools. Prior to this invention, a miniature ultrasound transducer as part of a colonoscope did not, to our knowledge, exist.

The feasibility of this procedure for safely improving the bowel prep is achieved when the entire colon segment is cleaned and inspected in about 20 minutes, or more or less, and histological scores of the colon tissue are less than 1.

One suitable transducer 22 for use with the present invention is available, with modification, from Vensa, LLC. The embodiment can be one of the following, but not limited to: 1) an ultrasound transducer outside a colonoscope; 2) a miniaturized transducer placed on the tip of a colonoscope (as an aftermarket product or integrated into the working components of a colonoscope); or 3) a flexible catheter that can be inserted into the accessory port of a standard colonoscope, or any combination of the above. The transducer 22 can have a broad range of frequency and power so as to allow the endoscopist to visualize the gastrointestinal mucosa in a variety of settings such as when the intestinal debris or fecal material contain organic or vegetable matter, blood, or other foreign objects. It can also be used to degrade, debulk, and resect intestinal tumors. When faced with gastrointestinal bleeding, such as a bleeding peptic ulcer or diverticular bleed that impairs adequate visualization, embodiments of the invention can be used to liquefy blood clots and suction it out of one or more lumens 24, and allow the endoscopist to find and treat the source of bleeding.

The present invention can address the above-listed weaknesses in the current methods of preparing the patient for a routine colonoscopy. It allows the physician to clean the colon during the colonoscopy examination itself. This can eliminate the need for undesirable bowel preparation prior to the examination. Embodiments of the invention are expected to lead to a much greater number of subjects undergoing screening colonoscopy and reduce the incidence of colon cancer-related deaths. In addition to improved health, the increase in the number of screenings can lead to cost-savings. Studies have shown that improving compliance from 50% to 80% for undergoing a colonoscopy can increase life-years gained to about 30 per 1,000 people screened. The screening strategy of the present invention can provide net savings by reducing a later need for expensive treatment such as chemotherapy. For example, of the estimated $21.1 billion spent in diagnosing and treating cancer among Medicare patients in 2004, colon cancer ranked the second costliest cancer.

Embodiments of the invention can also reduce average costs by avoiding the need for repeat visits, resulting from examinations that had to be prematurely stopped due to incomplete cleansing. As mentioned above, these delays and repeat visits due to improper colon preparation increases costs by about 20%. This represents a significant cost savings, as well. Reimbursement cost of a colonoscopy is about $2000 in a hospital setting and less if done in an outpatient clinic setting. Approximately 14.2 million colonoscopies are done per year in the United States. Even if used in only one million procedures, reducing costs by about 20% amounts to approximately $400 million per year in savings to the health care system.

In addition, embodiments of the invention can also reduce average time of the procedure. Methods according to the invention may increase time of the procedure by a few minutes. However, on average, the systems and methods save time. Procedures may take about 10 minutes when no stool is found and up to 30 minutes, or more or less, when remnants are found but the procedure can continue. The systems and methods reduce average time of procedures by eliminating long procedures that are caused by remnant solid stools.

To the best our knowledge, the present invention is the first method for improving the preparation for colonoscopies since its introduction forty years ago and leads to become the new standard for colonoscopies. In addition, embodiments of the low-profile low frequency transducer 22 is the first ultrasound transducer design capable of efficient ultrasound generation without the need for cymbical or horn type transducers.

To evaluate the possibility of using ultrasound to liquefy stools without the risk of damaging the intestinal lining, an available ultrasound transducer was used to attempt to liquefy dog stools and then calculated the Thermal Index “TI” and Mechanical Index “MI” as described below. It should be noted that higher frequencies (1000 KHz) does not generally affect viscosity of stools.

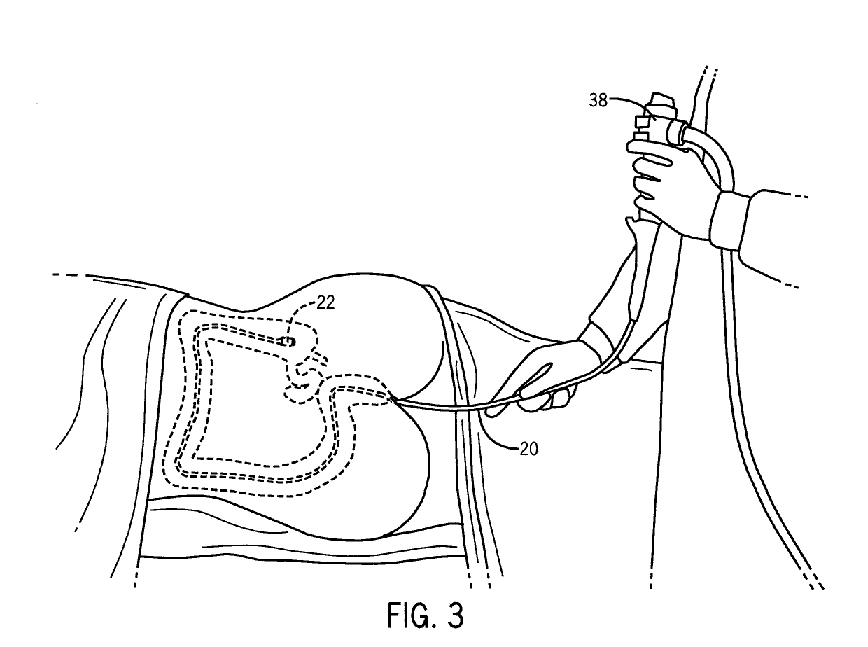

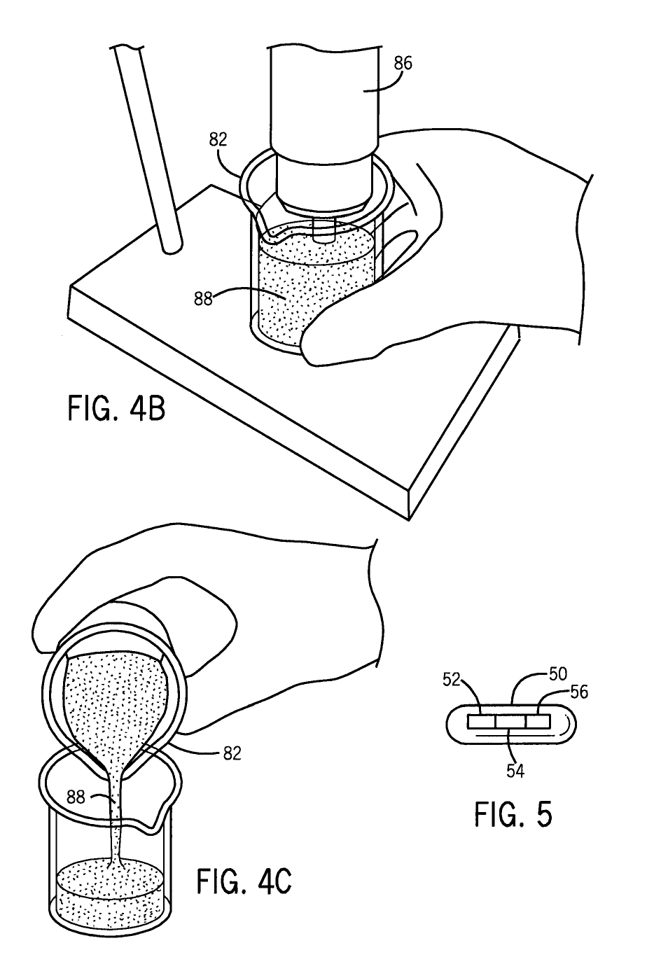

Referring to FIGS. 4A-4C, 20 mL of canine stools 80 of solid consistency was placed in a glass cup 82 and then filled with 40 mL of water 84, as shown in FIG. 4A. The amount of water placed is consistent with the volume of water used in a colonoscopy examination where an irrigator can put out approximately 300 mL of water per minute. An ultrasound transducer 86 (VCX-400, Sonics & Materials, Newtown, Conn.), operating at a frequency of 20 kHz equipped with a 25 mm diameter probe was immersed in the water without touching the stool and ultrasound 88 was applied at 40% duty cycle for one minute at an intensity of 3.2 W/cm2, as shown in FIG. 4B. The entire stool 80 was substantially liquefied 88 and poured easily from the glass cup 82, as shown in FIG. 4C. Using a validated visual description of stool (i.e., form, soft, loose, and liquid) that correlates with an objective measure of viscosity, the final product was rated as liquid. An identical procedure was done without ultrasound (passive control) and upon decanting the liquid, there was no detectable change in consistency after 10 minutes of exposure to water.

Mechanical Index is a standard measure of the acoustic output in a diagnostic ultrasound system. According to the Food and Drug Administration (FDA) for diagnostic obstetrics application, the MI may not exceed 1.9. In order to calculate the MI achieved in our preliminary experiment (f=20 kHz, 1=3.2 W/cm2), the intensity (I) and acoustic impedance of tissue (Z=1.5 1.5 MPa·s/m) was used in order to calculate the pressure (P), and we derived the following formula:

$$MI = \frac{P}{\sqrt{f}} = \frac{\sqrt{I \cdot Z}}{\sqrt{f}} = \frac{\sqrt{0.32 \cdot 1.56 e 6}}{\sqrt{20 e 3}} = 4.90 \qquad ( 1 )$$

Another standard measure is the Thermal Index. TI is intended to indicate the likely temperature rise that might be produced after long exposure. A larger TI value represents a higher risk of damage. For obstetrical applications, it has been global standard to keep the thermal index lower than one. The calculated soft-tissue thermal index (“Ts”) for the ultrasound intensity (I) and probe area (A) is:

$${T}_{S} = \frac{I \cdot A \cdot f}{210 e 3} = \frac{( 3.2 ) ( 4.91 ) ( 20 e 3 )}{210 e 3} = 1.49 \qquad ( 2 )$$

As shown in equation (1) and (2), the calculated MI is greater than 1.9 and Ts value achieved is greater than one. It should be noted that these standard measures are for diagnostic ultrasound, and generally in therapeutic ultrasound applications the MI and TI are multiple orders of magnitude greater as in physiotherapy and rehabilitation ultrasound devices. Additionally, for stool liquefaction, the ultrasound transducer tip may not be applied directly on the tissue as in diagnostic applications, and the water that may be continuously applied can significantly reduce the temperature increase. Embodiments of a device used in the present invention may have multiple configurations for frequency and intensity according to our dosimetry study. Also, the calculation for MI does not refer to the derating factor along the beam axis, which further reduces the MI and TI colon indexes. These are only preliminary feasibility results using equipment that was not designed and optimized for stool liquefaction.

Safety as well as efficacy was evaluated with embodiments of the present invention. An inventor of the present invention successfully obtained FDA approval using 20 kHz ultrasound (drug delivery through the skin) with feedback control at about 5 to 8 W/cm2 for 5-90 seconds and a probe diameter of 8 mm. This is greater ultrasound intensity than what was used in the preliminary data.

Building an effective small device requires efficiently turning electric power into acoustic power. To achieve this goal, an ultralow impedance approach developed by one of the inventors may be used. This approach may be used to optimize the ultrasound generator circuit 30, wiring 32 and transducer 22 to maximize energy transfer from the ultrasound generator 30 to the transducer 22. As detailed below, the transducer 22 may be fabricated, for example, from a piezoelectric material with high electro-mechanical efficiency, and the system may be housed 34 in a biocompatible material with very low acoustic losses (less than 5%, for example).

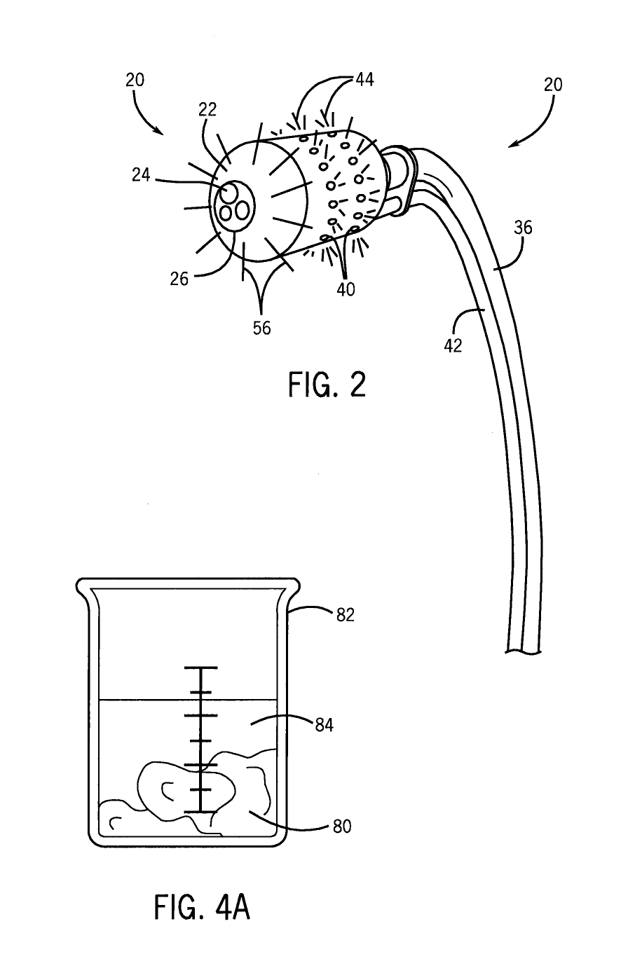

In some embodiments, the transducer 22 may be a probe that is about 1.5 cm in diameter and about 1 to 2 cm long, or more or less, that is inserted over the tip of a standard colonoscope 36 and connected to an ultrasound control console 38, which may be hand-held or computer based for example. In some embodiments, the transducer 22 may be fabricated from multiple flat 15-25 mm diameter lead-zirconate-titanate (PZT-4 or PZT-8 EBL Products Inc.) piezoelectric donuts. Wrap-tabs may be staked back-to-back to electrically connect the front and back surfaces of the piezoelectric from a single side. Alternating stacks of piezoelectrics lower the resonance impedance of the transducer 22. The stacks may be surrounded by air to improve electro-mechanical conversion in ultrasound production. In one embodiment, the piezoelectrics operate in their lateral modes of resonance and may be coupled mechanically to a custom aluminum housing 36 that acts as a resonating beam for ultrasound transmission at 20 kHz, although transmission may range at least from about 10 kHz to about 30 kHz, or more or less. The aluminum housing 36 may be designed to maintain a low-profile that may be easily inserted and removed from the colon. In one embodiment, ultrasound radiates perpendicular to the surface of the transducer tip of the colonoscope, and parallel to two water flow channel(s) 40 incorporated into the transducer. The water and electrical power to the transducer may be supplied via a thin, flexible conduit 42 running generally parallel to the colonoscope 20, as shown in FIG. 2. Water 44 is shown spraying out from the channels 40. In another embodiment, in addition to, or in place of the transducer 22 at the tip of the colonoscope, a transducer 46 may be housed at the water spray pump 48. Here, sonicated water 44 can be injected through the conduit 42 of a colonoscope and can liquefy stools without the need for a transducer at or near the tip of the colonoscope.

As seen in FIG. 5, an alternative embodiment is a wireless therapeutic ultrasound capsule 50 with physiologic impedance and pH sensors 52, 54 with its own energy source 56. After the capsule 50 is swallowed, it can detect when it has entered the colon and may automatically start sonification to liquefy stools. The capsule can also be used, under different ultrasound frequencies parameters, to alter intestinal permeability for medical therapy such as enhancing drug absorption.

In some embodiments, the colonoscope 20 may be combined with ultrasonic agitation of soft bristles 56 (see FIG. 2) to provide additional mechanical agitation to improve liquefaction at a lower intensity. In addition, the effect of water irrigation can be enhanced by causing acoustic cavitations in the water to provide micro-convective cavitation-nuclei to improve stool liquefaction.

The principles underlying the technology and construction of efficient ultrasound systems have been described elsewhere. In some embodiments of the present invention, we provide an ultralow output impedance ultrasound generator design, based off of a 16 to 32-MOSFET, surface mount components, printed circuit board (PCB) design, and its application in ultrasound-assisted colonoscopy. It is to be appreciated that the ultrasound energy may also be combined with other energies, such as laser and/or electricity, for example. In addition, the liquefaction properties of the miniature ultrasound transducer 22 can also be used in non-medical applications, such as a portable device for water treatment or desalination. For example, the use of ultrasound is able to degrade bacteria, salt, and other harmful organic matter so as to facilitate its filtration and removal for safe consumption.

The ultralow output impedance ultrasound generator 30 may be constructed on a double sided PCB, which may be designed and created using PCB123® Layout V2 software from Sunstone Circuits Inc. The PCB may have 16 to 32 N/P channel parallel MOSFET'S in a transistor-transistor logic (TTL) timing configuration to provide efficient voltage transfer from the generator 30 to the ultrasound transducer 22. An onboard microcontroller 60 capable of controlling ultrasound parameters and measuring output energy may be incorporated into the generator design. A user interface 62 and software 64 previously developed for monitoring acoustic energy, adjusting power, and modulating the ultrasound drive signal waveform may also be used and may be incorporated with the control console 38. The microcontroller 60 can be developed using modules that can be purchased from suppliers such as Digilent, Inc. and Idec Corp. In some embodiments, the microcontroller 60 may be interfaced with a waveform generator circuit 66 to replace a function generator or other timing source. The microcontroller 60 and user interface 62 may be programmed using WinAVR software, for example.

In one embodiment, the colonoscope 20 may have the following features:
Pulse width (0-1010 cycles) and drive signal frequency modulation (0-500 kHz) of the TTL timing signal. Embodiments of the invention may provide features for control of ultrasound excitation for stool liquefaction.
Automatic tuning features for a transducer with multiple harmonic drive and real-time onboard electrical power output measurement from the generator 30. This allows the low-frequency transducer 22 to lock into a controlled acoustic power output to prevent hysteresis in transducer resonance. Additionally, this feature allows one to monitor acoustic intensity/power once the transducer has been characterized with the power generation electronics.
Computer and/or onboard control of MOSFET switching power supply. The real-time feedback from an optional computer 68 provides consistent acoustic power/intensity. Additionally, the computer control maintains treatment regimes under defined parameters.
Generator and transducer overload monitor as part of the ultrasound generator circuit 30. Feedback in the electronics and transducer measures temperature and current jumps to prevent damage to both components. For example:

a. The ultrasound generator 30 may provide electrical output powers from about 0-180 Watts.

b. The surface area of the transducer may be about 9.42 cm2 with an estimated 85% efficient electrical to ultrasound conversion. Therefore, embodiments of the invention have the capability to provide (0.85×180/9.42)=16 w/cm2 of acoustic energy, or more or less, across its entire surface.

c. This acoustic power may be limited by using the above feedback control to a maximum of about 10 w/cm2 from the transducer.

The microprocessor controlled ultrasound generator 30 may operate off of standard power supplies and power the 20 kHz low frequency colonoscope transducer 22 over a range of power settings.

The ultrasonic pressure and intensity of the acoustic field for different voltage settings may be determined with a miniature (e.g., 1-mm diameter) omnidirectional reference hydrophone. For example, the transducer tipped colonoscope 20 can be submerged in a distilled-water tank (e.g., 30×30×85 cm) that is made almost completely anechoic by placing a 2 cm thick wall of sound absorbing rubber around its wall. The water in the tank is degassed to less than 2 ppm using a custom inline degassing device (Philips Research Inc.).

Precise, micromanipulator-controlled positioning of the hydrophone is performed using a computerized micropositioning system. Pressure waves detected by the hydrophone are recorded by a digitizing oscilloscope. For example, the scanning step size for each plane is 1 mm and the scanning area is 10×10 mm. Spatial peak-temporal peak intensity (ISPTP) is determined over each plane in 1 mm increments from the transducer face using the hydrophone, based on three scans of the transducer for a mean and standard deviation of the results.

The transducer 22 and electronic technology in the present invention has been well proven in many aspects of research. Embodiments of the present systems and methods provide sufficient ultrasound power to enhance the dissolution of stool in the colon. In some specific situations, additional power may be needed. For example, ultrasound attenuation is made by the aluminum housing 34 or imperfect acoustic coupling between the ultrasound transducer 22 and the stool 80. In this situation, high-current transformers may be incorporated, for example, DC-100 kHz, 1:2 and 1:4, to double or quadruple acoustic output from the device. Although this may increase output impedance of the electronics by a factor of 4 or 16, the output impedance is still in the range from about 0.16 ohms to about 0.8 ohms from the present invention, which is an acceptable value for the transducer. Both commercially available transformers and custom-wound transformers can be used.

When cooling is required, the ultrasound transducer can be maintained at a safe temperature by a water circulatory system that is straight forward to incorporate. Additionally, relatively low acoustic powers can provide rapid stool liquefaction.

The transducer 22 may be symmetric around the radial axis, radiating ultrasound in all directions. The transducer 22 can also be approximately 15 mm in diameter, for example. In some embodiments, the radial symmetry can be removed and limit ultrasound radiation to a single segment of the colonoscope 20. The transducer 22 may be positioned at the front of the colonoscope and focus ultrasound energy through the water stream 44, thereby providing additional ultrasound energy to assist the water stream 44 to liquefy stool. In some embodiments, the transducer 22 may be inserted through an accessory port of the colonoscope and pushed in front of the colonoscope to liquefy stool in the optical field of the colonoscope with the assistance of a water jet.

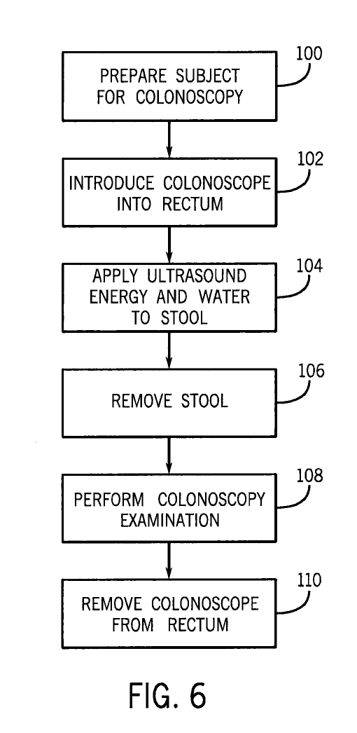

A method according to embodiments of the invention is set forth in FIG. 6. As indicated at process block 100, a subject may be premedicated and prepared for a colonoscopy. An embodiment of colonoscope 20 is introduced into the rectum and advanced proximally, as indicated at process block 102.

When solid stools are encountered, ultrasound energy is applied via the ultrasound transducer without touching the stools, and may include water irrigation to aid in the stool liquefaction process, as indicated at process block 104. Stool may be removed with applied suction 70 using conventional methods, such as until the entire mucosa of the colon segment can be seen well, and with minimal or no residual staining, small fragments of stool, or opaque liquid using a validated bowel prep scoring system, as indicated at process block 106. The colonoscopy may be performed by a single operator, and may be digitally recorded, as indicated at process block 108. When complete, the colonoscope 20 is removed and the subject is allowed to recover, as indicated at process block 110.

The foregoing has been a detailed description of illustrative embodiments of the invention. Various modifications and additions can be made without departing from the spirit and scope thereof. Furthermore, since numerous modifications and changes will readily occur to those skilled in the art, it is not desired to limit the invention to the exact construction and operation shown and described. For example, any of the various features described herein can be combined with some or all of the other features described herein according to alternate embodiments. While the preferred embodiment has been described, the details may be changed without departing from the invention, which is defined by the claims.

Finally, it is expressly contemplated that any of the processes or steps described herein may be combined, eliminated, or reordered. In other embodiments, instructions may reside in computer readable medium wherein those instructions are executed by a processor to perform one or more of processes or steps described herein. As such, it is expressly contemplated that any of the processes or steps described herein can be implemented as hardware, software, including program instructions executing on a computer, or a combination of hardware and software. Accordingly, this description is meant to be taken only by way of example, and not to otherwise limit the scope of this invention.

I claim:

1. A medical device comprising:
a flexible tube having an operable end insertable into a body cavity and a control end;
an ultrasound transducer housing positioned at or near the operable end of the flexible tube, the housing including an ultrasound transducer to generate ultrasound energy and at least one water flow channel to deliver water to the body cavity; and
an ultrasound generator circuit to control the ultrasound transducer, the ultrasound generator circuit coupled to the ultrasound transducer.

2. The device of claim 1 further including a source of suction to withdraw the water delivered to the body cavity.

3. The device of claim 1 wherein the ultrasound generator circuit is coupled to a control console, the control console positioned at or near the control end.

4. The device of claim 1 wherein the water flow channel and electrical power extend from the control end to the ultrasound transducer housing.

5. The device of claim 1 wherein the ultrasound transducer housing is inserted over the operable end of the flexible tube.

6. The device of claim 1 wherein the flexible tube is inserted into an accessory port of a standard colonoscope.

7. The device of claim 1 wherein ultrasound generator circuit includes a transducer overload monitor.

8. The device of claim 1 wherein the ultrasound generator provides an electrical output from about zero watts to about 180 watts.

9. The device of claim 1 wherein the ultrasound transducer produces about 16 w/cm2 of acoustic energy.

10. The device of claim 1 wherein the ultrasound transducer operates in a range of about 10 kHz to about 30 kHz.

11. The device of claim 1 wherein the water is used to cool the ultrasound transducer.

12. The device of claim 1 wherein ultrasound energy is focused through the water stream.

13. The device of claim 1 further including soft bristles to provide additional mechanical agitation.

14. The device of claim 1 further including causing acoustic cavitations in the water to provide micro-convective cavitation-neclei to improve stool liquefaction

15. A method comprising:
introducing a flexible tube having an operable end insertable into a gastrointestinal tract, the flexible tubing including an ultrasound transducer housing positioned at or near the operable end of the flexible tube, the housing including an ultrasound transducer to generate ultrasound energy and at least one water flow channel to deliver water to the gastrointestinal tract;
applying ultrasound energy to stool located in the gastrointestinal tract;
delivering water through the at least one water flow channel to the gastrointestinal tract;
liquefying the stool via the ultrasound energy and water; and
removing the liquefied stool from the gastrointestinal tract.
